(19) United States
(12) Patent Application Publication
(10) Pub. No.: US 2025/0099288 A1
(43) Pub. Date: Mar. 27, 2025

(54) BLADDER RELIEF SYSTEM WITH ADHESIVE IMPREGNATED USER INTERFACE AND METHOD OF USE

(71) Applicant: Mark R. Harvie, Colchester, VT (US)

(72) Inventor: Mark R. Harvie, Colchester, VT (US)

(21) Appl. No.: 18/891,391

(22) Filed: Sep. 20, 2024

Related U.S. Application Data
(60) Provisional application No. 63/584,439, filed on Sep. 21, 2023.

(51) Int. Cl. A61F 5/44; A61F 5/441; A61F 5/443; A61F 5/455

(52) U.S. Cl. CPC A61F 5/4404; A61F 5/441; A61F 5/443; A61F 5/4556

(57) ABSTRACT

This invention relates generally to bladder relief devices. This invention is a device and method of use that incorporates the features of existing technologies with a user interface attachment means comprised of a reusable pliable gel that is impregnated with an adhesive together with a pressure relief balloon system and a proprietary Hydro-block or similar substance filter or air vent allowing air to flow into a urine cup collection means while simultaneously blocking the urine from exiting except through the intended drainage tubing to a fluid storage bag. A user of this invention can use it while in any position, such as: while the user is moving, working or exercising; the user is in reclined positions; the user is in lying positions; the user is in seated positions; the user is in standing positions; or the user is in zero gravity such as in outer space.

### CROSS REFERENCE TO RELATED APPLICATION

This application claims priority from the Applicant's Provisional Patent Application No. 63/584,439 which was filed on Sep. 21, 2023.

### STATEMENT AS TO RIGHTS TO INVENTIONS MADE UNDER FEDERALLY SPONSORED RESEARCH OR DEVELOPMENT

Not applicable.

### REFERENCE TO A SEQUENCE LISTING, A TABLE OR A COMPUTER PROGRAM LISTING APPENDIX SUBMITTED ON A COMPACT DISC

Not Applicable.

### FIELD OF THE INVENTION

This invention relates generally to bladder relief devices. This invention is a bladder relief system device that incorporates the features of existing patented technologies disclosed in this application for letters patent with a user interface attachment means comprised of a pliable gel that is impregnated with an adhesive together with a proprietary Hydro-block or similar substance filter or air vent that allows air to flow into the urine cup collection means while simultaneously blocking the urine from exiting except through the intended drainage tubing to the urine or fluid storage bag.

### BACKGROUND ART

The use of human urinary collection and disposal systems improve pilot and aircraft safety. is known in the prior art. For example, U.S. Pat. No. 4,886,508 (Washington, 1989) discloses a ladies' external catheter assembly, however this device does not use a vacuum pump for drainage or utilize a moisture sensor. Also U.S. Pat. No. 4,610,675 (Triunfol, 1986) teaches a device for collecting fluid discharged from female organs that is designed solely for incontinent women, not female aircrew members and the design includes a pad, vacuum pump and liquid sensor, however, the pad is more invasive because it is formed of plastic and has ridges to move the labia to an open position for free flow of liquid. The vacuum pump of the Triunfol patent is powered by an electrical outlet and does teach battery operation of these devices. In U.S. Pat. No. 5,662,631 (Marx, Sep. 2, 1997) a male external catheter assembly with vacuum retention is disclosed wherein a male external catheter attachment incorporates a vacuum or a means to produce reduced pressure to aid in installing and keeping the device in place. U.S. Pat. No. 5,499,977 (Marx, Mar. 19, 1996) teaches another form of male external catheter with vacuum assist utilizing a rubber bulb that functions as a vacuum. As such, the basic concept of bladder discharge collection systems and their use are disclosed.

Urinary incontinence affects more than 13 million Americans in community and institutional settings. Thirty-eight percent of non-institutionalized patients older than 60 years of age experienced urinary incontinence, and almost 50 percent of institutionalized patients. The annual costs of bladder control problems in the United States for people older than 65 years of age was estimated at $26.3 billion in 1995, or $3,565 per affected person. Many incontinent males use commercially available diapers, which cannot contain urine from multiple urinations, and become heavy and uncomfortable when wet. Significant advances, however, have been made in bladder relief devices as taught in U.S. Pat. No. 6,706,027 (Harvie, Mar. 16, 2004) (automatic or semi-automatic bladder relief system utilizing a pump and collection bag); U.S. Pat. No. 6,918,899 (Harvie, Jul. 19, 2005) (automatic or semi-automatic bladder relief system incorporating an inflatable urine collection means that may be deflated after use); U.S. Pat. No. 7,131,964 (Harvie, Nov. 7, 2006) (automatic or semi-automatic bladder relief system incorporating: an inflatable urine collector deflatable after use, disposable batteries, disposable urine storage bag with an isolated cleaning fluid chamber with directional valve and pump system that automatically rinses and sanitizes the pump, hoses, collection means, and a failsafe device in the event of system failure being detachable from the pump with a stored extendable portion forming a seal with user's body allowing urination into the bag without urine splash-back and a storage bag containing absorbent polymer crystals to absorbing the deposited urine); U.S. Pat. No. 7,141,043 (Harvie, Nov. 28, 2006) (automatic or semi-automatic bladder relief and hydration system incorporating an inflatable urine collection means that may be deflated after use, a dual chambered back pack to store urine and potable fluid and/or liquid food, disposable batteries integrated into a disposable urine storage bag, urine storage with a cleaning fluid chamber with a directional valve and pump system that automatically rinses and sanitizes the pump, hoses, collection means and the user with cleaning fluid stored in the cleaning fluid chamber, the disposable urine storage bag containing absorbent polymer crystals to absorbing deposited urine while in use); U.S. Pat. No. 7,335,189 (Harvie, Feb. 26, 2008) (automatic or semi-automatic bladder relief system incorporating a power unit with a battery that is integrated into a disposable urine storage bag or into a disposable female pad or male cup); U.S. Pat. No. 7,875,011 (Harvie, Jan. 25, 2011) (integrating an automatic or semi-automatic bladder relief system with Cooling, Heating, Gas, Hydration and a Nutrition Chem-Bio Suit Connectivity System used connecting various life function support systems in Chemical-Biological Protective Suits); U.S. Pat. No. 9,788,992 (Harvie, Oct. 17, 2017) (hydro-block venting system to prevent urine backflow in condom catheters); and U.S. Pat. No. 11,026,829 (Harvie, Jun. 8, 2021) (integrating a hydro-block venting system to prevent urine backflow in automatic or semi-automatic bladder relief systems and integrating indwelling catheters in both male and female user configurations).

Despite the advances that have been made in the prior art one problem has persisted with bladder relief devices. That is leakage. Because of the location on the body of the urethra of both males and females and the wide variety of body types and differing anatomies it has proven to be very difficult to interface a bladder relief device with a human body that does not leak, especially when the device is being used while the user is active, such as with pilots or other users actively moving that have no access to sanitary facilitates and depend upon a bladder relief device. Leakage is the primary cause of user reluctance to use a bladder relief device. The purpose of using a bladder relief device in the first place is so that a user can urinate without wetting themselves or their garments so if a user cannot have confidence that they will remain dry then why bothering using the device in the first place.

The prior art bladder relief systems, while effective in aiding those suffering with incontinence or otherwise have no access to sanitary facilities, as disclosed above, each have a number of significant problems such as:
1) Attachment by adhesive which is difficult to obtain an effective seal without channeling issues occurring and causing a serious skin irritation and damage upon removal for many users;
2) Effective adhesive applications generally require the skin to be dry and clean shaven
3) Most medical grade adhesives are only effective for application once and cannot be reused rendering the bladder relief device useless if not properly adhered to the body since the adhesive cannot be reused;
4) Medical grade adhesives have very little shear strength and fail under minimal stress from movement or body sweat;
5) Difficulty in applying and removing the bladder relief device without injuring the user;
6) Difficulty in securely attaching the bladder relief device to a user such that it remains functional, despite activity and varied conditions of the human genitalia; and
7) Accumulation of urine that causes irritation and urinary tract and other types of infections.

It is known in the art that most complications that occur with bladder relief devices are when the device is either used incorrectly or used for a longer period of time than intended. The most common complications that can occur are:
Skin irritation or urinary tract or bladder infections.
Constant wet skin can become soft and worn away by the external catheter.
Allergic reactions to the materials can develop as well.

Thus, while there are a number of prior art attempts to solve the problem of patients remaining wet while wearing these devices and developing infections, etc., none of these devices are satisfactory solutions. Therefore, there is a great need for a bladder relief device that is easily donned and remains securely in place without leaking regardless of the activity level of the user. Furthermore, to solve the problems of the prior art devices this bladder relief device must also effectively facilitate the aspiration of backwashed urine that may accumulate between the genitalia and the device that is unable to drain from the direct urine collection means. My invention is designed to solve these problems that exist in the current art.

### DISCLOSURE OF THE INVENTION

### Purpose of Invention

The purpose of my invention is to provide a bladder relief device that will permit a user to removably adhere the device to their body such that it forms a fluid tight seal against their body such that urine cannot escape from the device and wet the user or their garments. Additionally, the purpose of my invention is to provide hydro-block air venting to the device to prevent backwashing of urine thereby further preventing a leakage event by facilitating the removal of urine away from the body. The device will permit air to enter into the device by means of a Hydro-block, or similar substance, air vent that allows air to flow into the external component of the device while blocking the urine from exiting except through the drain as intended. This invention will allow the urine to completely drain out of the urine collection cup completely away from the user's skin without leakage outside of the device.

### Description of Problems Solved by Invention

There are currently no bladder relied devices that will fit a user securely enough to allow for reasonable physical activity of a user without leakage that will also permit air to enter into the device and thereby allow any backwashed urine to escape, thereby preventing such adverse medical conditions as skin irritation, maceration of the genitalia, urinary tract infections (UTI), ischemia and edema or urethral obstruction. The current invention incorporates a proprietary Gel Adhesive Interface and at least one Hydro-block filter or air vent that allows air to flow into the Fluid Containment Vessel while blocking the urine from exiting except through the device to a collection bag. This invention will allow the urine to completely drain out of the device and any attached hose without backwash and leakage and ultimately away from the user's skin, unlike current art bladder relief devices which leak and permit urine to accumulate and remain in contact with the user's skin and urethra.

This invention will allow the bladder relief device to be small in size allowing for a unique and multiple sizing for each anatomy. The small size makes it possible for a user to wear the system under normal clothing without fear of leakage and wetting their clothing. Also with the addition of my patented bladder relief sensor activated pump (U.S. Pat. No. 8,997,348), the urine is completely drained out of the bladder relief device away from the user's skin leaving them dry at all times no matter what physical orientation their body is in, (i.e. standing, walking, climbing, sitting, reclined, lying flat on back, raised legs or in a hospital bed, lying on either side, lying on stomach in fact even upside down). This will result in significant reduction in skin ulcers, maceration and UTI which are currently all major problems and are a significant cost to the healthcare system for inpatient hospital care, nursing home long term care and most importantly home health care resulting in significant reduction in complications and wound care caused by urinary incontinence.

The most critical function of the invention disclosed in these letters patent is the reusable gel adhesive interface that provides a leakproof connection of the device to a user and the aeration of the device to prevent backwash further reducing potential leakage and/or medical complications from prolonged urine contact.

### BRIEF DESCRIPTION OF INVENTION

My invention is a bladder relief system which incorporates some of the features of my prior patented technologies disclosed above with a user interface attachment means comprised of a pliable gel that is impregnated with an adhesive together with a proprietary Hydro-block or similar substance filter or air vent that allows air to flow into the urine cup collection means while simultaneously blocking the urine from exiting except through the intended drainage tubing to the urine or fluid storage bag. The adhesive impregnated pliable gel can be reattached or moved on the body several times without losing its adhesive properties. Because the adhesive permeates the entire gel the adhesive has about the same shear strength as the gel it impregnates. To further facilitate an effective leakproof seal to the user's skin the adhesive impregnated gel when formed has a portion of the gel specifically designed to more effectively adhere to the perineum of a user thereby facilitating the optimum leakproof interface to the body. The gel is sufficiently pliable that it will conform to any user's peculiar anatomy with a leakproof seal. The gel and the adhesive being specifically engineered such that it will form a leakproof seal without requiring any preparation of the skin regardless of hair or other skin conditions while simultaneously allowing for pain free removal without solvents or residue. The adhesive impregnated gel being sufficiently hydrophobic that fluids such as urine or sweat or hair will not affect the interface adhesive bond to the user's skin. This invention will allow the urine without leakage from the device to completely drain out of the system and any attached hose and ultimately away from the user's skin.

Unlike the prior art my invention places pre-foamed flat gel sheets into a 3-D mold and injects foam into it to form a leakproof user interface. The raised temperatures that happen during the foam expansion and curing process create a very strong bond. Also, my invention has the added advantage over prior art of being compact and discrete. Many potential users in need of a bladder relief device are disinclined to use available devices because of the size of the devices and the inability to conceal the fact that they need and use such a device, which for the majority of people is very embarrassing. My invention fits comfortably and is very discreet in its size such that others will not know a user is wearing the device.

Prior art devices are typically dependent on body position in order to be able to evacuate all the user's urine from the device and not leak. Also, the prior art devices generally are not capable of being used in: reclined positions; lying positions; or in zero gravity and for the most part can only be effectively used in a seated or standing position. My invention's functionality and leakproof quality is unaffected by the position of the user. A user of my invention can use it while in any position, such as: while the user is moving, working or exercising; the user is in reclined positions; the user is in lying positions; the user is in seated positions; the user is in standing positions; or the user is in zero gravity such as in outer space.

### BRIEF DESCRIPTION OF THE DRAWINGS

FIG. 1 is an exploded perspective side view of the Fabric Layer Adhered embodiment of the Bladder Relief System with Adhesive Impregnated User Interface.

FIG. 2 is a top view of the Fabric Layer Adhered embodiment of the Bladder Relief System with Adhesive Impregnated User Interface also depicting the Fluid Sensors.

FIG. 3 is side view of the Fabric Layer Adhered embodiment of the Bladder Relief System with Adhesive Impregnated User Interface also depicting the Fluid Sensors and Hydro-Block Filter.

FIG. 4 is bottom view of the Fabric Layer Adhered embodiment of the Bladder Relief System with Adhesive Impregnated User Interface.

FIG. 5 is an exploded perspective side view of the Inflatable Pad Adhered embodiment of the Bladder Relief System with Adhesive Impregnated User Interface.

FIG. 6 is a perspective side view of the Fabric Layer Adhered embodiment and/or Inflatable Pad Adhered embodiment of the Bladder Relief System with Adhesive Impregnated User Interface functionally connected to a User Controller further functionally connected to an air pump and fluid pump and further functionally connected to a Fluid Storage Bag.

FIG. 7 is a right side sagittal median plane view of the pelvis of a human female with the Fabric Layer Adhered embodiment of the Bladder Relief System with Adhesive Impregnated User Interface adhered to the body depicting the adhered contact of an adhesive gel layer with the mons pubis, perineum and anus of the body with the Inflatable Pad being inflated thereby causing the Fluid Containment Vessel to extend and form a urine receptacle.

### BEST MODES FOR CARRYING OUT THE INVENTION

### I. Preferred Embodiments

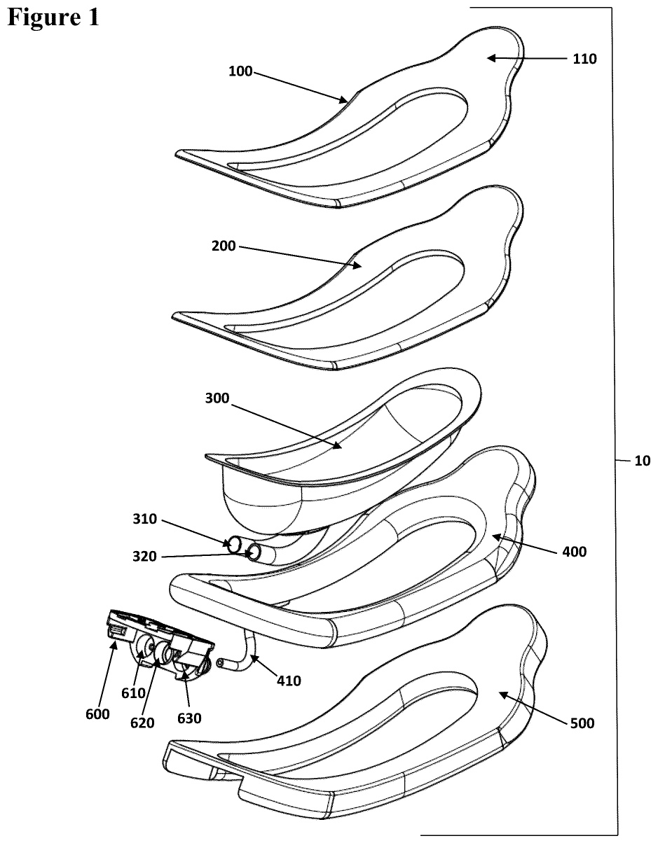

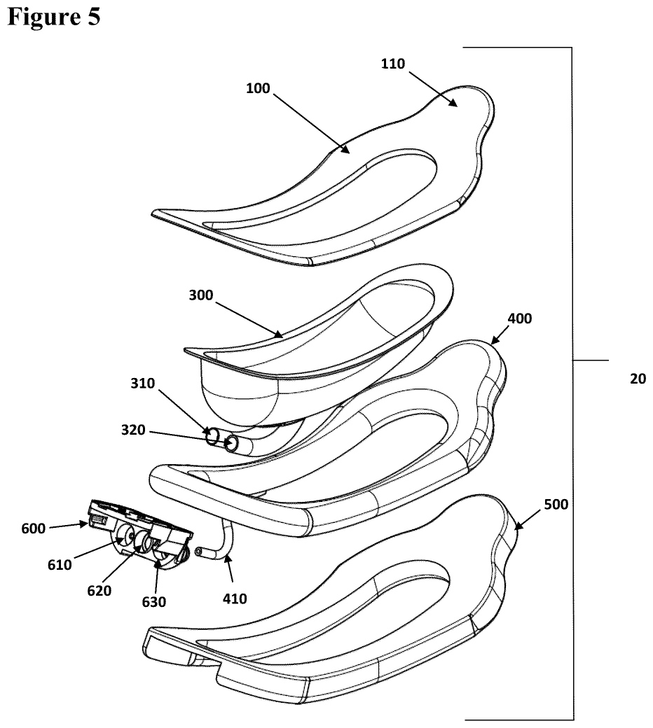

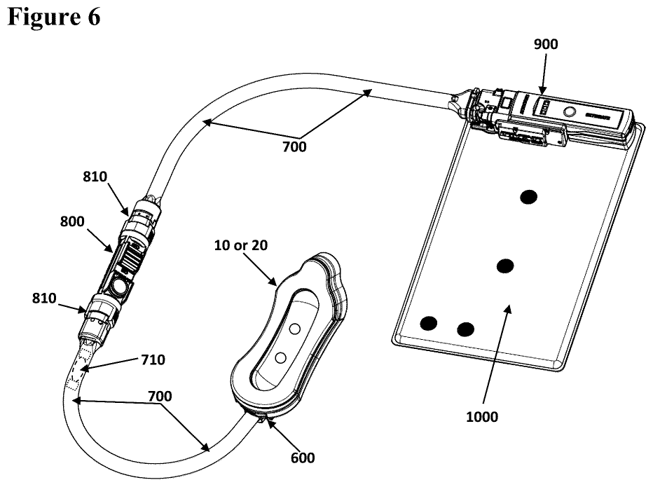

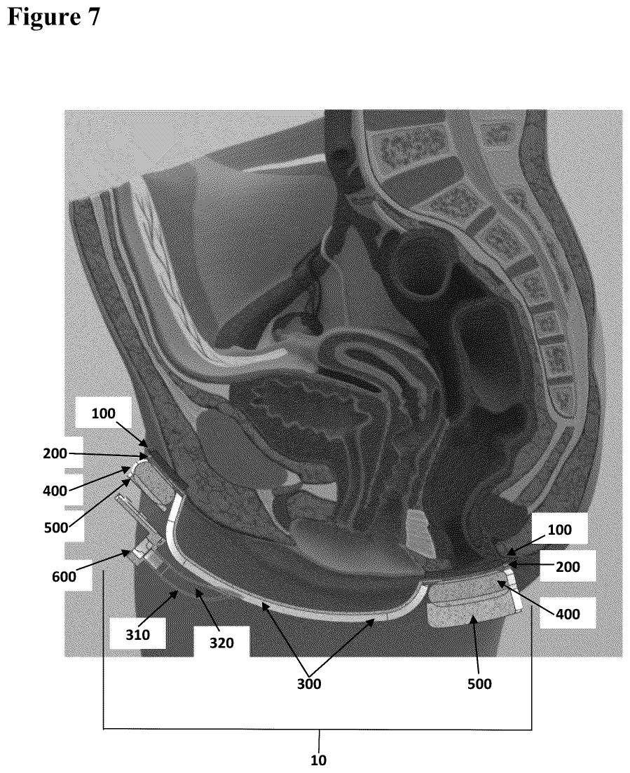

With reference now to the drawings, and in particular to FIGS. 1-7 thereof, are depicted a Bladder Relief System with Adhesive Impregnated User Interface which is engineered to allow a user to interface with a bladder relief device that effectively seals to the body such that when a user urinates the urine is effectively collected and transported from the device to a storage means without leaking fluids at the interface. The leak proof feature of this invention is accomplished by incorporating a flexible Gel Adhesive Interface with a Perineum Contact Area that permits the device to be conformed to the unique contours of their body and effectively adhere to the user's perineum and area of the body surrounding the genitalia. FIGS. 1-4 depict the new and novel Fabric Layer Adhered embodiment of the Bladder Relief System with Adhesive Impregnated User Interface. FIG. 5 depict the new and novel Inflatable Pad Adhered embodiment of the Bladder Relief System with Adhesive Impregnated User Interface. FIG. 6 depict a new and novel Fabric Layer Adhered embodiment and/or Inflatable Pad Adhered embodiment of the Bladder Relief System with Adhesive Impregnated User Interface that is functionally connected to a User Controller further functionally connected to an air pump, fluid pump and battery pack within an Air Pump, Fluid Pump and Battery Housing which is further functionally connected to a Fluid Storage Bag. FIG. 7 depict a new and novel Fabric Layer Adhered embodiment and/or Inflatable Pad Adhered embodiment of the Bladder Relief System with Adhesive Impregnated User Interface that is functionally attached to a user depicted in a right side sagittal median plane view of the pelvis of a human female. The figure depicts the adhered contact of a gel adhesive interface with the mons pubis, perineum and anus of the body with the Inflatable Pad being inflated thereby causing the Fluid Containment Vessel to extend and form a urine receptacle.

FIG. 1 depicts an exploded perspective view of the Fabric Layer Adhered embodiment of the Bladder Relief System with Adhesive Impregnated User Interface (10) comprised of a Gel Adhesive Interface (100). The device has at least one Fluid Sensor (330) disposed in the Fluid Containment Vessel (300) which is in electronic communication with an air pump, fluid pump and battery pack functionally contained within an Air Pump, Fluid Pump and Battery Housing (900) to draw expelled urine from the Fluid Containment Vessel (300) to a user selected storage or disposal means such as the Fluid Storage Bag (1000) depicted in FIG. 6. The Fabric Layer Adhered embodiment of the Bladder Relief System with Adhesive Impregnated User Interface (10) is further comprised of Hydro-Block Filter (640).

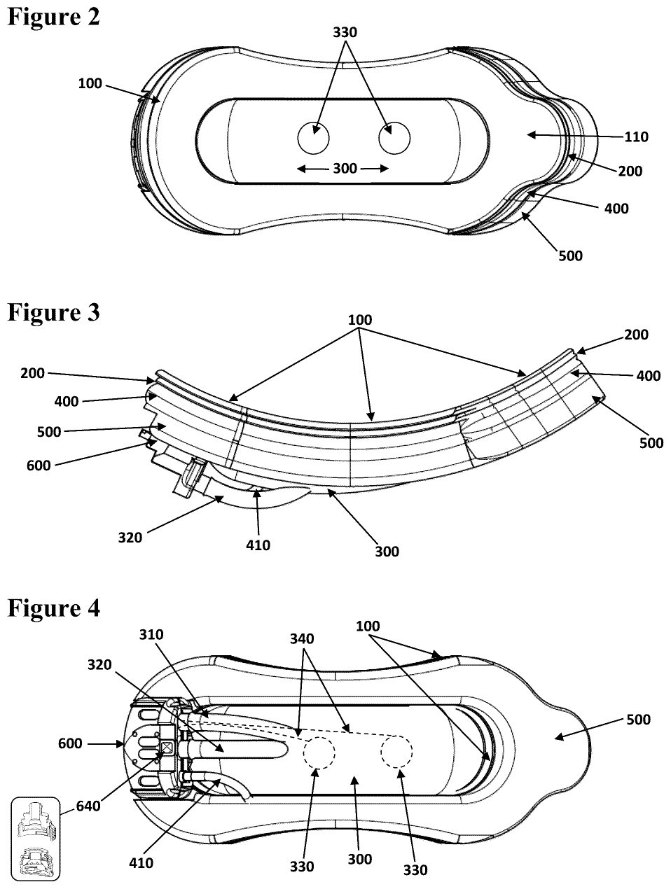

FIGS. 2, 3 and 4 depict, top side and bottom views of the Fabric Layer Adhered embodiment of the Bladder Relief System with Adhesive Impregnated User Interface (10).

FIG. 5 depicts an exploded view of the Inflatable Pad Adhered embodiment of the Bladder Relief System with Adhesive Impregnated User Interface (20) wherein the Gel Adhesive Interface (100) rather than be attached to a fabric layer it is adhered to the Inflatable Pad (400). The device has at least one Fluid Sensor (330) disposed in the Fluid Containment Vessel (300) which is in electronic communication with an air pump, fluid pump and battery pack functionally contained within an Air Pump, Fluid Pump and Battery Housing (900) to draw expelled urine from the Fluid Containment Vessel (300) to a user selected storage or disposal means such as the Fluid Storage Bag (1000) depicted in FIG. 6. The Inflatable Pad Adhered embodiment of the Bladder Relief System with Adhesive Impregnated User Interface (20) is further comprised of Hydro-Block Filter (640).

FIG. 6 depicts both the Fabric Layer Adhered embodiment of the Bladder Relief System with Adhesive Impregnated User Interface (10) and/or the Inflatable Pad Adhered embodiment of the Bladder Relief System with Adhesive Impregnated User Interface (20) functionally connected to a User Controller (800) utilizing a Quick Disconnect Coupling (810) and an air pump, fluid pump and battery contained within an Air Pump, Fluid Pump and Battery Housing (900) by means of an Air Hose, Fluid Drain Tube and Wiring Conduit (700) wherein urine excreted by a user will be transported and stored in the Fluid Storage Bag (1000) and a Pressure Relief Balloon (710).

FIG. 7 depicts the Fabric Layer Adhered embodiment and/or Inflatable Pad Adhered embodiment of the Bladder Relief System with Adhesive Impregnated User Interface (10) that is functionally attached to a user depicted in a right side sagittal median plane view of the pelvis of a human female. The figure depicts the adhered contact of the Gel Adhesive Interface (100) with the mons pubis, perineum and anus of the body with the Inflatable Pad (400) being inflated thereby causing the Fluid Containment Vessel (300) to extend and form a urine receptacle.

### GENERAL DESCRIPTION OF REFERENCE NUMERALS IN THE DESCRIPTION AND DRAWINGS

Any actual dimensions listed are those of the preferred embodiment. Actual dimensions or exact hardware details and means may vary in a final product or most preferred embodiment and should be considered means for so as not to narrow the claims of the patent.

### LIST AND DESCRIPTION OF COMPONENT PARTS OF THE INVENTION

(10) Fabric Layer Adhered embodiment of the Bladder Relief System with Adhesive Impregnated User Interface
(20) Inflatable Pad Adhered embodiment of the Bladder Relief System with Adhesive Impregnated User Interface
(100) Gel Adhesive Interface
(110) Perineum Contact Area
(200) Fabric Layer
(300) Fluid Containment Vessel
(310) Fluid Drain Tube
(320) Sensor Wiring Conduit
(330) Fluid Sensor
(340) Fluid Sensor Wiring
(400) Inflatable Pad
(410) Inflation Air Hose
(500) Foam Comfort Pad
(600) Connection Block
(610) Inflation Air Connection Port
(620) Fluid Drain Connection Port
(630) Sensor Wiring Connection Port
(640) Hydro-Block Filter
(700) Air Hose, Fluid Drain Tube and Wiring Conduit
(710) Pressure Relief Balloon
(800) User Controller
(810) Quick Disconnect Coupling
(900) Air Pump, Fluid Pump and Battery Housing
(1000) Fluid Storage Bag

### Detailed Description of the Preferred Embodiments

1) Fabric Layer Adhered embodiment of the Bladder Relief System with Adhesive Impregnated User Interface.

The Fabric Layer Adhered embodiment of the Bladder Relief System with Adhesive Impregnated User Interface (10) depicted in FIGS. 1, 2, 3, 4 and 7 is manufactured and comprised of the following components in their respective functional relationships:

The Fabric Layer Adhered embodiment of the Bladder Relief System with Adhesive Impregnated User Interface (10) as depicted on FIGS. 1-4, 6 and 7 is formed in a size and shape capable of fitting between the legs and over the genitalia of a user and forming a fluid tight seal around the genitalia and against the perineum and anus, the mons pubis and the adjacent groin areas for use by securing the device to a user by means of the Gel Adhesive Interface (100). A fluid tight seal against the body of a user is further facilitated by the Perineum Contact Area (110) that is disposed on the Gel Adhesive Interface (100) providing a larger surface area to adhere to the body as well as make applying pressure to the Gel Adhesive Interface (100) easier to more effectively adhere the Gel Adhesive Interface (100) to the body.

The Gel Adhesive Interface (100) formed is by: 1) selecting a pliable gel substrate material, preferably of medical grade, that is sufficiently flexible to conform and adapt to the conditions present in the groin area of user such that the gel will not lose contact with a user's skin where it was placed in position for use; 2) selecting an adhesive, preferably of medical grade and hydrophilic, that will adhere to human skin in a leakproof seal despite sweat or hair being present when applied and will release from the skin with reasonable force and will not damage the skin or leave a residue once removed; 3) selecting an enzyme capable of causing the pliable gel substrate material to set and form the gel with the pliable properties needed for use with this device; 4) combining into a thorough mixture the pliable gel substrate, the adhesive and the enzyme wherein the quantity of adhesive is sufficient to impregnate the gel once set and meet the specific desired adherence strength to form a leak proof user interface; and 5) pour the mixture into a shape determined mold or in a sheet for later cutting to size and shape to a desired thickness of the gel thereby forming the Gel Adhesive Interface (100) depicted in FIGS. 1-4, 6 and 7. In the Fabric Layer Adhered embodiment of the Bladder Relief System with Adhesive Impregnated User Interface (10) the gel substrate, adhesive and enzyme mixture are poured into the mold or sheet over a fabric material comprising the Fabric Layer (200) such that the fibers imbed within and become part of the gel once the gel sets thereby making a strong leakproof seal to the Fabric Layer (200).

As depicted in FIGS. 1-4, 6 and 7 the Fabric Layer (200) is comprised of a fabric of generally synthetic composition and fluid resistant properties. Once the Gel Adhesive Interface (100) is adhered to the Fabric Layer (200), the Fabric Layer (200) is then bonded with a fluid leakproof seal to the Fluid Containment Vessel (300) and the Inflatable Pad (400) after the Fluid Containment Vessel (300) and the Inflatable Pad (400) have first been bonded together with a fluid tight bond. The opposite side of the Inflatable Pad (400) from the bonded Fluid Containment Vessel (300) is then fluid tight bonded to the Foam Comfort Pad (500) The Fluid Containment Vessel (300) and the Inflatable Pad (400) being formed of a sufficiently pliable material such as silicone, urethane or the like.

As depicted in FIGS. 1-4, 6 and 7 the Fluid Containment Vessel (300) forms a vessel within which a user urinates once the Inflatable Pad (400) is inflated. The presence of the urine causes the Fluid Sensors (330) to transmit a signal through the Fluid Sensor Wiring (340) thereby activating the fluid pump in the Air Pump, Fluid Pump and Battery Housing (900) which when activated forms a suction in the fluid hose contained in the Air Hose, Fluid Drain Tube and Wiring Conduit (700) which draws urine out of the Fluid Containment Vessel (300) through the Fluid Drain Tube (310) functionally attached thereto. The fluid pump in the Air Pump, Fluid Pump and Battery Housing (900) then deposits the drawn urine into a Fluid Storage Bag (1000) for later disposal.

When a user is desirous of urinating they activate the device by means of the User Controller (800). The activation provides electrical current from the batteries in the Air Pump, Fluid Pump and Battery Housing (900) to activate the air pump in the Air Pump, Fluid Pump and Battery Housing (900) and provide current available to the Fluid Sensors (330). Once activated the air pump in the Air Pump, Fluid Pump and Battery Housing (900) inflates the Inflatable Pad (400) with air which in turn causes the attached Fluid Containment Vessel (500) to extend to its designed operational shape forming a vessel capable of receiving and containing urine flow from a user once they commence urination. Disposed on the internal surface of the Fluid Containment Vessel (300) is at least one Fluid Sensor (330) capable of urine sensing once a user begins to urinate and thereby activate the fluid pump in the Air Pump, Fluid Pump and Battery Housing (900) either by electronic means such as radio, WiFi, or by the direct cabling of three interconnected segments of Fluid Sensor Wiring (340) contained in the Sensor Wiring Conduit (320) that brings current from the battery to and through the sensor connected by the Sensor Wiring Connection Port (630) of the Connection Block (600) and a corresponding port of the Quick Disconnect Coupling (810) functionally attached to the User Controller (800).

Once a user has placed the device over the genitalia and it is attached to the user as heretofore described the user may pass urine into the device. During the process of urination gravity and excretion pressure from the user's bladder and urethra will cause the urine to flow into the Fluid Containment Vessel (300) and then be drawn away from the genitalia by the activated pump or vacuum means through the Fluid Drain Tube (310) and away from the user into a user selected collection or disposal means such as the Fluid Storage Bag (1000) depicted in FIG. 6. The urine will not be able to accumulate around the genitalia because the Hydro-block Filter (640) by preventing air locks by providing free air flow into and out of the Fluid Drain Tube (310) thereby causing the urine to completely drain away from the user's genitalia and ultimately discharge through three interconnected segments of Fluid Drain Tube (310) connected by the Fluid Drain Connection Port (620) of the Connection Block (600) and a corresponding port of the Quick Disconnect Coupling (810) functionally attached to the User Controller (800) and ultimately into a user selected storage means such as the Fluid Storage Bag (1000) depicted in FIG. 6.

The proper pumping and evacuation of the urine is facilitated by the Pressure Relief Balloon (710) which prevents air and fluid locks in the system that could prevent the proper pumping and/or draining of urine from the system. FIG. 6 depicts the Pressure Relief Balloon (710). As depicted in FIGS. 6 the Pressure Relief Balloon (710) is inserted within the middle section of the three Fluid Drain Tubes (310) thereby becoming part of the Fluid Drain Tube (310) permitting liquid to flow therethrough. The Pressure Relief Balloon (710) is composed of a material with elastic properties such as polyurethane. In order for the Fluid Sensors (330) to activate the fluid pump the Fluid Sensors (330) must be in contact with the user's urine during a urination event. Once a user commences urination the force of the urination causes the urine to flow into the Fluid Drain Tube (310) however the air in the Fluid Drain Tube (310) and the air in the proximity of the Fluid Sensor (330) prevent the Fluid Sensor (330) from having adequate contact with the user's urine to activate the Fluid Sensor (330) and thereby start the fluid pump housed in the Air Pump, Fluid Pump and Battery Housing (900).

Once the user's urine passes into the Pressure Relief Balloon (710) portion of the Fluid Drain Tube (310) contained within the Air Hose, Fluid Drain Tube and Wiring Conduit (700) the elastomeric properties of the Pressure Relief Balloon (710) provide an expandable and retractable vessel for the air that is being compressed by the urine flow as the Pressure Relief Balloon (710) begins to fill with urine such that sufficient air is forced out of the liquid hose contained within the Air Hose, Fluid Drain Tube and Wiring Conduit (700) and the area surrounding the Fluid Sensors (330) to permit the Fluid Sensor (330) to have sufficient contact with the user's urine to be activated and in turn start the fluid pump housed in the Air Pump, Fluid Pump and Battery Housing (900). The power necessary to activate the air pump and fluid pump housed in the Air Pump, Fluid Pump and Battery Housing (900) are the batteries functionally contained therein. The Pressure Relief Balloon (710) will be in its collapsed state prior to user urination and the Pressure Relief Balloon (710) will be in its expanded state during a user's urination with about 60 ml of urine contained therein.

Once a user finishes urinating the Fluid Sensor (330) will thereafter not sense the presence of urine since the user's urine has been drawn away by the fluid pump housed in the Air Pump, Fluid Pump and Battery Housing (900). The fluid pump housed in the Air Pump, Fluid Pump and Battery Housing (900) is programmed to continue sucking urine from the Fluid Drain Tube (310) after the cessation of urination and deactivation of the Fluid Sensor (330) for a sufficient period of time to evacuate the urine from the entire system and thereby restoring the Pressure Relief Balloon (710) to its pre-urination collapsed state. Once this evacuation cycle is complete the fluid pump housed in the Air Pump, Fluid Pump and Battery Housing (900) will shut off and stop pumping having pumped all the urine from the urination cycle of the user to a user selected storage means such as the Fluid Storage Bag (1000) depicted in FIG. 6.

2) Inflatable Pad Adhered embodiment of the Bladder Relief System with Adhesive Impregnated User Interface.

The Inflatable Pad Adhered embodiment of the Bladder Relief System with Adhesive Impregnated User Interface (20) as depicted in FIG. 5 functions in the identical manner utilizing all of the same identical component parts as the Fabric Layer Adhered embodiment of the Bladder Relief System with Adhesive Impregnated User Interface (10) depicted in FIGS. 1, 2, 3, 4, 6 and 7, except that the Inflatable Pad Adhered embodiment of the Bladder Relief System with Adhesive Impregnated User Interface (20) does not utilize a Fabric Layer (200) as depicted in the Fabric Layer Adhered embodiment of the Bladder Relief System with Adhesive Impregnated User Interface (10) detailed in FIGS. 1-4, 6 and 7. In the Inflatable Pad Adhered embodiment of the Bladder Relief System with Adhesive Impregnated User Interface (20) the gel substrate, adhesive and enzyme mixture are poured into a mold or sheet to form the Gel Adhesive Interface (100) that is then bonded with a fluid tight seal to the Inflatable Pad (400). In this embodiment the user will secure the device to their body, as well as use and maintain the device in the same exact manner as the Fabric Layer Adhered embodiment of the Bladder Relief System with Adhesive Impregnated User Interface (10).

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. For example, there are a wide variety of hydrophobic and visco-elastic gel substrates, such as styrene, butadiene, SBS, silicone, UV curable acrylates and urethane, etc. and it would be obvious to anyone trained in the art to use any one of these or other gel substrates to practice my invention. Similarly, it would be obvious to substitute or mix various adhesives such as dual hydrogen bond network adhesives and epoxies etc. to use as the adhesive component of the adhesive gel interface disclosed in my invention. Thus, the previous description is not intended to be limited to the implementations shown herein but is to be accorded the widest scope consistent with the principles and novel features disclosed herein. The invention should therefore not be limited by the above described embodiment, method, and examples, but by all embodiments and methods within the scope and spirit of the invention. Accordingly, the scope of the invention should be determined not by the embodiments illustrated, but by the claims and their legal equivalents which accompany this application.

Having described my invention, I claim:

1. A bladder relief system with adhesive impregnated user interface comprised of:
a fluid depository means wherein the fluid depository means is comprised of:
a gel adhesive interface wherein the gel adhesive interface is comprised of a hydrophilic adhesive capable of adhering to human skin forming a fluid leakproof seal that when applied to a user's skin forms a fluid leakproof barrier between the user's genital area and the rest of the user's body;
a fabric layer attached to the gel adhesive interface such that fibers disposed on the surface of the fabric layer imbed within the gel adhesive interface thereby forming a fluid leakproof bond between the fabric layer and the gel adhesive interface;
a fluid containment vessel;
an inflatable pad attached with a fluid leakproof bond to the fluid containment vessel thereby forming a bonded inflatable pad and fluid containment vessel;
the bonded inflatable pad and fluid containment vessel attached with a fluid leakproof bond to the fabric layer wherein the fluid containment vessel is capable of receiving urine discharged from a user when the gel adhesive interface is adhered to the user's skin forming a fluid leakproof barrier between the user's genital area and the rest of the user's body;
a foam comfort pad attached with a fluid leakproof bond to the bonded inflatable pad and fluid containment vessel;
a fluid transport means attached to the bonded inflatable pad and fluid containment vessel and to the fluid depository means capable of transporting a user's urine that has been discharged into the fluid containment vessel;
the fluid transport means being comprised of a fluid pump, a power source and at least one fluid drain tube;
a fluid storage means attached to the fluid transport means wherein the fluid storage means is capable of receiving and storing a user's urine that has been transported to the fluid storage means by the fluid transport means and is comprised of a leakproof storage bag;
an air pump operatively attached to the power source;
the air pump also operatively attached to the inflatable pad by at least one air hose such that when the air pump is activated the inflatable pad inflates from a generally flat condition to an inflated condition thereby forming with the inflatable pad an area where urine discharged by a user can pool generally away from contact with the user's body and then the pooled urine can be transported by the fluid transport means and upon deactivation of the air pump the inflatable pad discharges the pumped air by means of compression caused by the user's seated body weight and returns to the generally flat condition;
a pressure relief balloon attached to the fluid drain tube such that as air is displaced and compressed above ambient air pressure in the fluid containment vessel by a user's discharged urine the pressure relief balloon which is in a collapsed state then inflates and thereby decompresses the increased air pressure in the fluid containment vessel;
a hydro-block filter air vent attached to the fluid drain tube wherein the hydro-block filter air vent permits ambient air to enter into or escape from the fluid drain tube thereby preventing air locks in the fluid drain tube by providing free air flow into and out of the fluid drain tube.
a fluid sensor attached to the fluid containment vessel capable of detecting the presence of a user's urine in the fluid depository means and electronically communicating the presence of urine to the air pump and the fluid pump;
the fluid sensor upon the detection of the user's urine activates the air pump thereby inflating the inflatable pad by means of the air pump;
the fluid sensor upon the detection of the user's urine activates the fluid pump thereby pumping the user's detected urine from the fluid containment vessel to the leakproof storage bag by means of the fluid transport means;
the pressure relief balloon capable of regulating liquids in the fluid drain tube such that the fluid sensor will detect the presence of liquids without air bubble interference;
the air pump and the fluid pump being deactivated once the fluid sensor no longer detects a user's urine in the fluid containment vessel; and
the pressure relief balloon returning to a collapsed state once a user's urine is pumped out of the fluid containment vessel by the fluid pump.

2. A bladder relief system with adhesive impregnated user interface comprised of:
a fluid depository means wherein the fluid depository means is comprised of:
a gel adhesive interface wherein the gel adhesive interface is comprised of a hydrophilic adhesive capable of adhering to human skin forming a fluid leakproof seal that when applied to a user's skin forms a fluid leakproof barrier between the user's genital area and the rest of the user's body;
a fluid containment vessel;
an inflatable pad attached with a fluid leakproof bond to the fluid containment vessel thereby forming a bonded inflatable pad and fluid containment vessel;
the bonded inflatable pad and fluid containment vessel attached with a fluid leakproof bond to the gel adhesive interface wherein the fluid containment vessel is capable of receiving urine discharged from a user when the gel adhesive interface is adhered to the user's skin forming a fluid leakproof barrier between the user's genital area and the rest of the user's body;
a foam comfort pad attached with a fluid leakproof bond to the bonded inflatable pad and fluid containment vessel;
a fluid transport means attached to the bonded inflatable pad and fluid containment vessel and to the fluid depository means capable of transporting a user's urine that has been discharged into the fluid containment vessel;
the fluid transport means being comprised of a fluid pump, a power source and at least one fluid drain tube;
a fluid storage means attached to the fluid transport means wherein the fluid storage means is capable of receiving and storing a user's urine that has been transported to the fluid storage means by the fluid transport means and is comprised of a leakproof storage bag;
an air pump operatively attached to the power source;
the air pump also operatively attached to the inflatable pad by at least one air hose such that when the air pump is activated the inflatable pad inflates from a generally flat condition to an inflated condition thereby forming with the inflatable pad an area where urine discharged by a user can pool generally away from contact with the user's body and then the pooled urine can be transported by the fluid transport means and upon deactivation of the air pump the inflatable pad discharges the pumped air by means of compression caused by the user's seated body weight and returns to the generally flat condition;
a pressure relief balloon attached to the fluid drain tube such that as air is displaced and compressed above ambient air pressure in the fluid containment vessel by a user's discharged urine the pressure relief balloon which is in a collapsed state then inflates and thereby decompresses the increased air pressure in the fluid containment vessel;
a hydro-block filter air vent attached to the fluid drain tube wherein the hydro-block filter air vent permits ambient air to enter into or escape from the fluid drain tube thereby preventing air locks in the fluid drain tube by providing free air flow into and out of the fluid drain tube.
a fluid sensor attached to the fluid containment vessel capable of detecting the presence of a user's urine in the fluid depository means and electronically communicating the presence of urine to the air pump and the fluid pump;
the fluid sensor upon the detection of the user's urine activates the air pump thereby inflating the inflatable pad by means of the air pump;
the fluid sensor upon the detection of the user's urine activates the fluid pump thereby pumping the user's detected urine from the fluid containment vessel to the leakproof storage bag by means of the fluid transport means;
the pressure relief balloon capable of regulating liquids in the fluid drain tube such that the fluid sensor will detect the presence of liquids without air bubble interference;
the air pump and the fluid pump being deactivated once the fluid sensor no longer detects a user's urine in the fluid containment vessel; and
the pressure relief balloon returning to a collapsed state once a user's urine is pumped out of the fluid containment vessel by the fluid pump.

3. The bladder relief system with adhesive impregnated user interface of claim 1, wherein the gel adhesive interface is further comprised of:
a pliable gel substrate material of medical grade sufficiently flexible to conform and adapt to the conditions present in the groin area of a user such that the gel adhesive interface will not lose contact with a user's skin once placed in position for use;
a medical grade and hydrophilic adhesive capable of adhering to human skin in a leakproof seal despite sweat or hair being present when applied and will release from the skin with reasonable force and will not damage the skin or leave a residue once removed;
an enzyme capable of causing the pliable gel substrate material to set and form the gel adhesive interface with the pliable properties needed for use by a user;
the gel adhesive interface being formed by the combination into a thorough mixture of the pliable gel substrate, the adhesive and the enzyme wherein the quantity of adhesive is sufficient to impregnate the gel once set and meet the specific desired adherence strength to form a leak proof user interface; and
the gel adhesive interface size and shape being formed by pouring the mixture of the pliable gel substrate, the adhesive and the enzyme into a shape determined mold or in a sheet for later cutting to size and shape to a user desired thickness.

4. The bladder relief system with adhesive impregnated user interface of claim 2, wherein the gel adhesive interface is further comprised of:
a pliable gel substrate material of medical grade sufficiently flexible to conform and adapt to the conditions present in the groin area of a user such that the gel adhesive interface will not lose contact with a user's skin once placed in position for use;
a medical grade and hydrophilic adhesive capable of adhering to human skin in a leakproof seal despite sweat or hair being present when applied and will release from the skin with reasonable force and will not damage the skin or leave a residue once removed;
an enzyme capable of causing the pliable gel substrate material to set and form the gel adhesive interface with the pliable properties needed for use by a user;
the gel adhesive interface being formed by the combination into a thorough mixture of the pliable gel substrate, the adhesive and the enzyme wherein the quantity of adhesive is sufficient to impregnate the gel once set and meet the specific desired adherence strength to form a leak proof user interface; and
the gel adhesive interface size and shape being formed by pouring the mixture of the pliable gel substrate, the adhesive and the enzyme into a shape determined mold or in a sheet for later cutting to size and shape to a user desired thickness.

5. The bladder relief system with adhesive impregnated user interface of claim 1 wherein the hydro-block filter air vent is attached to the gel adhesive interface.

6. The bladder relief system with adhesive impregnated user interface of claim 2 wherein the hydro-block filter air vent is attached to the gel adhesive interface.

7. The bladder relief system with adhesive impregnated user interface of claim 1 wherein the fluid containment vessel is collapsible and maintains a collapsed state until the inflatable pad is inflated by means of the air pump while a user is discharging urine into the fluid containment vessel wherein the fluid containment vessel will be caused to become and remain in an uncollapsed state by the inflation of the inflatable pad and thereafter returning to a collapsed state once the user's urine has been evacuated from the fluid containment vessel by the fluid transport means.

8. The bladder relief system with adhesive impregnated user interface of claim 2 wherein the fluid containment vessel is collapsible and maintains a collapsed state until the inflatable pad is inflated by means of the air pump while a user is discharging urine into the fluid containment vessel wherein the fluid containment vessel will be caused to become and remain in an uncollapsed state by the inflation of the inflatable pad and thereafter returning to a collapsed state once the user's urine has been evacuated from the fluid containment vessel by the fluid transport means.

9. A method of using bladder relief system with adhesive impregnated user interface of claim 1 consisting of the steps of:
selecting a size of the bladder relief system with adhesive impregnated user interface such that when the gel adhesive interface is adhered to the user's body the bladder relief system with adhesive impregnated user interface adequately covers the genital area of the user and can adequately be adhered to the body such that excreted urine from the user will not leak out of the bladder relief system with adhesive impregnated user interface;
cleaning the genital area of the user;
cleaning the area around the genitals where the gel adhesive interface is adhered to the user's body;
positioning the bladder relief system with adhesive impregnated user interface over the user's genital area to facilitate the complete flow of urine into the fluid containment vessel;
pressing firmly the gel adhesive interface against the skin of the user to form a complete leakproof seal to the skin that surrounds the genital area;
selecting an air pump;
connecting the air pump to the power source;
connecting the air pump to the inflatable pad;
connecting the air pump to the fluid sensor;
selecting a fluid pump;
connecting the fluid pump to the power source and at least one fluid drain tube;
connecting the fluid pump to the bonded inflatable pad and fluid containment vessel and to the fluid depository means such that when activated the fluid pump will draw urine from the fluid containment vessel and discharge the user's urine into the fluid depository means;
connecting the fluid pump to the fluid sensor;
turning the fluid pump and the air pump on such that when the fluid sensor detects the presence of the user's urine the fluid pump will turn on and draw the urine away from the fluid containment vessel through the fluid drain tube then through the fluid pump to be discharged in the fluid depository means and simultaneously the air pump will inflate the inflatable pad;
urinating into the bladder relief system with adhesive impregnated user interface that is attached to the user whenever the user feels the urge to urinate;
removing the bladder relief system with adhesive impregnated user interface from the user by peeling off the attached gel adhesive interface from the user;
disposing of the user's urine that was deposited in the fluid depository means;
cleaning and storing the bladder relief system with adhesive impregnated user interface for future use.
